(19) United States
(12) Patent Application Publication
(10) Pub. No.: US 2023/0193211 A1
(43) Pub. Date: Jun. 22, 2023

(54) METHOD FOR PRODUCING THREE-DIMENSIONAL CELL STRUCTURE, AND THREE-DIMENSIONAL CELL STRUCTURE

(71) Applicant: TOPPAN Inc., Taito-ku (JP)

(72) Inventors: Rii MORIMURA, Taito-ku (JP); Isana NADA, Taito-ku (JP); Shiro KITANO, Taito-ku (JP)

(73) Assignee: TOPPAN Inc., Taito-ku (JP)

(21) Appl. No.: 18/171,862

(22) Filed: Feb. 21, 2023

Related U.S. Application Data
(63) Continuation of application No. PCT/JP2021/030300, filed on Aug. 19, 2021.

(30) Foreign Application Priority Data
Aug. 21, 2020 (JP) 2020-140134

(51) Int. Cl. C12N 5/071

(52) U.S. Cl. CPC C12N 5/069; C12N 2513/00; C12N 2533/50; C12N 2533/90

(57) ABSTRACT

A method for producing a three-dimensional cell structure includes preparing a mixture of a cationic substance, an extracellular matrix component, a polyelectrolyte, and a cell population including endothelial cells and mouse-derived stromal cells, which exclude mouse-derived endothelial cells, collecting a cell aggregate from the mixture, and culturing a collected cell aggregate to obtain a three-dimensional cell structure.

### CROSS-REFERENCE TO RELATED APPLICATIONS

The present application is a continuation of International Application No. PCT/JP2021/030300, filed Aug. 19, 2021, which is based upon and claims the benefits of priority to Japanese Application No. 2020-140134, filed Aug. 21, 2020. The entire contents of all of the above applications are incorporated herein by reference.

### BACKGROUND OF THE INVENTION

### Field of the Invention

The present invention relates to methods for producing a three-dimensional cell structure, and to three-dimensional cell structures.

### Discussion of the Background

In recent years, in the field of regenerative medicine, drug assay systems that require an environment close to that of the living body, and the like, the superiority of using three-dimensional cell structures that are three-dimensionally organized over using cells grown on plates has been demonstrated. Accordingly, various techniques have been developed to construct three-dimensional cell structures in vitro.

The inventors of the present application have previously developed techniques for producing a three-dimensional cell structure, comprising a step of obtaining a mixture in which cells are suspended in a solution comprising at least a cationic buffer solution, an extracellular matrix component, and a polyelectrolyte; a step of collecting the cells from the obtained mixture to form a cell aggregate on a substrate; and a step of culturing the cells to obtain a three-dimensional cell structure (see, for example, JP 6639634 B).

### SUMMARY OF THE INVENTION

According to an aspect of the present invention, a method for producing a three-dimensional cell structure includes preparing a mixture of a cationic substance, an extracellular matrix component, a polyelectrolyte, and a cell population including endothelial cells and mouse-derived stromal cells, which exclude mouse-derived endothelial cells, collecting a cell aggregate from the mixture, and culturing a collected cell aggregate to obtain a three-dimensional cell structure.

According to another aspect of the present invention, a three-dimensional cell structure includes a cell population comprising endothelial cells and mouse-derived stromal cells, the mouse-derived stromal cells excluding mouse-derived endothelial cells, a cationic substance, an extracellular matrix component, and a polyelectrolyte. A ratio of the endothelial cells to the mouse-derived stromal cells in the cell population is 1.0%-50%.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows fluorescence micrographs of slices of respective three-dimensional cell structures, taken in Experimental Example 1.

FIG. 2 is a graph showing the results of measuring maximum values of thicknesses of the respective three-dimensional cell structures based on FIG. 1.

FIG. 3 shows fluorescence micrographs showing the results of staining vascular endothelial cells of the respective three-dimensional cell structures in Experimental Example 1.

FIG. 4 shows fluorescence micrographs of a slice of a three-dimensional cell structure, taken in Experimental Example 2.

### DESCRIPTION OF THE EMBODIMENTS

The embodiments will now be described with reference to the accompanying drawings, wherein like reference numerals designate corresponding or identical elements throughout the various drawings.

### <Method for Producing Three-Dimensional Cell Structure>

In an embodiment, the present invention provides a method for producing a three-dimensional cell structure, comprising:

a first step of performing a process of:
obtaining a mixture of a cell population, a cationic substance, an extracellular matrix component, and a polyelectrolyte, the cell population comprising mouse-derived stromal cells, the mouse-derived stromal cells excluding mouse-derived endothelial cells; and
collecting one or more cell aggregates from the obtained mixture; and

a second step of performing a culturing of the collected one or more cell aggregates to obtain one or more three-dimensional cell structures,

the cell population comprising endothelial cells in addition to the mouse-derived stromal cells.

The production method of the present embodiment is, in other words, a method for producing a three-dimensional cell structure, comprising:

a first step of performing a process of:
obtaining a mixture of a cell population, a cationic substance, an extracellular matrix component, and a polyelectrolyte, the cell population comprising endothelial cells and mouse-derived stromal cells, the mouse-derived stromal cells excluding mouse-derived endothelial cells; and
collecting one or more cell aggregates from the obtained mixture; and

a second step of performing a culturing of the collected one or more cell aggregates to obtain one or more three-dimensional cell structures.

In the present specification, the term “three-dimensional cell structure” refers to a three-dimensional cell aggregate. The three-dimensional cell structure produced by the production method of the present embodiment comprises at least endothelial cells and mouse-derived stromal cells, the mouse-derived stromal cells excluding mouse-derived endothelial cells.

Examples of uses of the three-dimensional cell structure include, but are not limited to, living tissue models and solid cancer models. Examples of living tissue models include skin, hair, bone, cartilage, tooth, cornea, blood vessel, lymphatic vessel, heart, liver, pancreas, nerve, and esophagus models. Examples of solid cancer models include stomach cancer, esophageal cancer, bowel cancer, colon cancer, rectal cancer, pancreatic cancer, breast cancer, ovarian cancer, prostate cancer, renal cell cancer, and liver cancer models.

For example, for analysis of the behavior of immune cells relative to cancer cells, or the like, the three-dimensional cell structure may further comprise immune cells. In this case, all the cells constituting the three-dimensional cell structure are preferably syngeneic.

The form of the three-dimensional cell structure is not particularly limited, and may be, for example, a three-dimensional cell structure formed by culturing cells within a cell culture insert, or a three-dimensional cell structure formed by culturing cells within a scaffold composed of a natural biopolymer, such as collagen, or a synthetic polymer, or a cell aggregate (spheroid), or a sheet-like cell structure.

The present inventors found that, when a three-dimensional cell structure is produced using mouse-derived cells, adding endothelial cells to a cell population used allows suppression of decrease in thickness of the three-dimensional cell structure over time, and completed the present invention.

The production method of the present embodiment can suppress decrease in thickness of a three-dimensional cell structure over time even when the three-dimensional cell structure is produced using mouse-derived cells. The extent of suppression of decrease in thickness of a three-dimensional cell structure over time may be, for example, such that: a first three-dimensional cell structure sample, which is selected from the one or more cell aggregates immediately after production as the one or more three-dimensional cell structures, has a first portion obtained therefrom as a first slice along a predetermined first straight line that passes through a first center of gravity of a top surface of the first three-dimensional cell structure sample; a second three-dimensional cell structure sample, which is selected from the one or more cell aggregates on the fifth day since production as the one or more three-dimensional cell structures, has a second portion obtained therefrom as a second slice along a predetermined second straight line that passes through a second center of gravity of a top surface of the second three-dimensional cell structure sample; and each of the first and second slices has a maximum width, the maximum width of the second slice being greater than or equal to 50% of that of the first slice. Here, the maximum width of the second slice on the fifth day since production is, in other words, the maximum width of a second slice which is a second portion cut away from a second three-dimensional cell structure sample, which is selected from the one or more cell aggregates on the fifth day since start of the culturing, along a predetermined second straight line that passes through a second center of gravity of a top surface of the second three-dimensional cell structure sample. Further, the maximum width of the first slice immediately after production is, in other words, the maximum width of a first slice which is a first portion cut away from a first three-dimensional cell structure sample, which is selected from the one or more cell aggregates immediately after start of the culturing, along a predetermined first straight line that passes through a first center of gravity of a top surface of the first three-dimensional cell structure sample. Here, “immediately after production” may refer to “when 5 minutes to 72 hours has elapsed since the start of culture of one or more cell aggregates in step (C)” or “after one day (preferably, when 24 hours has elapsed) since the start of culture of one or more cell aggregates in step (C)”. Further, “on the fifth day since production” may refer to “on the fifth day (preferably, when 96 hours has elapsed) since culture of one or more cell aggregates started in step (C).

Here, the width of a slice obtained from a three-dimensional cell structure along a straight line passing through the center of gravity of the top surface thereof is the width of a slice obtained from the central part of a three-dimensional cell structure. The shape of the three-dimensional cell structure depends on the container used to produce the three-dimensional cell structure; when the three-dimensional cell structure is produced using, for example, a cylindrical-shaped cell culture insert, it becomes a cylindrical shape. In this case, the shape of the three-dimensional cell structure when viewed from the top surface is a circle, and the center of gravity when viewed from the top surface is the center of the circle. The shape of the three-dimensional cell structure is not limited to a cylindrical shape and can be any shape according to the purpose. Specifically, for example, a polygonal prism shape such as a triangular prism shape and a quadrangular prism shape can be exemplified.

The production method of the present embodiment includes step (A) of obtaining a mixture of a cell population, a cationic substance, an extracellular matrix component, and a polyelectrolyte, the cell population comprising endothelial cells and mouse-derived stromal cells, the mouse-derived stromal cells excluding mouse-derived endothelial cells; step (B) of collecting one or more cell aggregates from the obtained mixture; and step (C) of performing a culturing of the collected one or more cell aggregates to obtain one or more three-dimensional cell structures. Each step will be described below.

First, in step (A), a cell population comprising mouse-derived stromal cells (except for mouse-derived endothelial cells) and endothelial cells, a cationic substance, an extracellular matrix component, and a polyelectrolyte are mixed to obtain a mixture thereof. In step (A), the mixture is preferably obtained in an aqueous medium.

stromal cell is a generic name for cells constituting a supportive tissue for epithelial cells. Examples of stromal cells include fibroblasts and smooth muscle cells. Whether a cell is an stromal cell can be determined by the morphology of the cell as observed under a microscope or by the expression of marker molecules on the cell.

Markers of fibroblasts include Fibroblast growth factor receptor (FGFR) 1, FGFR2, FGFR3, CD90, and vimentin. Markers of smooth muscle cells include actin, desmin, calponin, and SM22.

In the production method of the present embodiment, mouse-derived cells are used as stromal cells. These stromal cells may be used singly or in combination of two or more. Further, stromal cells originating from a species other than mice may be used in addition to mouse-derived stromal cells. Examples of species other than mice include humans, monkeys, dogs, cats, rabbits, pigs, cows, and rats.

Examples of the endothelial cells include vascular endothelial cells and lymphatic endothelial cells; vascular endothelial cells are preferable. The origin of the endothelial cells is not particularly limited, and they may originate from, for example, a human, monkey, dog, cat, rabbit, pig, cow, mouse, or rat. Among them, mouse-derived endothelial cells are preferable.

Whether a cell is an endothelial cell can be determined by the morphology of the cell as observed under a microscope or by the expression of marker molecules on the cell.

Markers of vascular endothelial cells include CD31, VEGFR-2, and Tie-2/Tek. Markers of lymphatic endothelial cells include podoplanin, LYVE-1, PROX-1, and VEGFR-3.

In the production method of the present embodiment, it is preferable that stromal cells be fibroblasts and endothelial cells be vascular endothelial cells.

In the production method of the present embodiment, the ratio of the number of endothelial cells to the number of stromal cells (except for endothelial cells) in a cell population is preferably 1.0% or more and 50% or less, and may be 1.0% or more and 20% or less, 1.5% or more and 20% or less, or 1.5% or more and 10% or less. As will be described in the Examples, with the proportion of endothelial cells in the above range, decrease in thickness of a three-dimensional cell structure over time tends to be suppressed even when the three-dimensional cell structure is produced using mouse-derived cells.

The cell population may comprise cells other than both mouse-derived stromal cells (except for mouse-derived endothelial cells) and endothelial cells. Examples of such cells include somatic cells originating from bone, muscle, internal organs, nerve, brain, bone, skin, blood, or the like, germ cells, induced pluripotent stem cells (iPS cells), embryonic stem cells (ES cells), tissue stem cells, and cancer cells. Examples of the somatic cells originating from blood include immune cells such as lymphocyte, neutrophil, macrophage, and dendritic cells. Examples of the cancer cells include cells of stomach cancer, esophageal cancer, bowel cancer, colon cancer, rectal cancer, pancreatic cancer, breast cancer, ovarian cancer, prostate cancer, renal cell cancer, and liver cancer.

The cells constituting the cell population may be primary cells or cultured cells such as subcultured cells and cell lines.

As the cationic substance, any positively charged substance can be used, as long as it does not adversely affect the cell growth and the formation of cell aggregates. Examples of the cationic substance include, but are not limited to, cationic buffer solutions, such as tris-hydrochloric acid buffer solution, tris-maleic acid buffer solution, bis-tris buffer solution, and HEPES; ethanolamine; diethanolamine; triethanolamine; polyvinylamine; polyallylamine; polylysine, polyhistidine; and polyarginine. Among them, cationic buffer solutions are preferable, and tris-hydrochloric acid buffer solution is more preferable.

The concentration of the cationic substance in step (A) is not particularly limited, as long as it does not adversely affect the cell growth and the formation of cell aggregates. The concentration of the cationic substance used in the present embodiment is preferably 10 to 100 mM, and may be, for example, 20 to 90 mM, 30 to 80 mM, 40 to 70 mM, or 45 to 60 mM.

When a cationic buffer solution is used as the cationic substance, the pH of the cationic buffer solution is not particularly limited, as long as it does not adversely affect the cell growth and the formation of cell aggregates. The pH of the cationic buffer solution used in the present embodiment is preferably 6.0 to 8.0. For example, the pH of the cationic buffer solution used in the present embodiment may be 7.0, 7.1, 7.2, 7.3, 7.4, 7.5, 7.6, 7.7, 7.8, 7.9, or 8.0. The pH of the cationic buffer solution used in the present embodiment is more preferably 7.2 to 7.6, and even more preferably 7.4.

As the extracellular matrix component, any component that constitutes an extracellular matrix (ECM) can be used, as long as it does not adversely affect the cell growth and the formation of cell aggregates. Examples of the extracellular matrix component include, but are not limited to, collagen, laminin, fibronectin, vitronectin, elastin, tenascin, entactin, fibrillin, proteoglycan, and modified forms or variants thereof. The extracellular matrix component may be used singly or in combination of two or more.

Examples of the proteoglycan include, but are not limited to, chondroitin sulfate proteoglycan, heparan sulfate proteoglycan, keratan sulfate proteoglycan, and dermatan sulfate proteoglycan. The extracellular matrix component is preferably collagen, laminin, or fibronectin; collagen is particularly preferable.

The concentration of the extracellular matrix component is preferably, but is not particularly limited to, more than 0 mg/mL and less than 1.0 mg/mL, as long as it does not adversely affect the cell growth and the formation of cell aggregates. The concentration of the extracellular matrix component may be 0.005 mg/mL or more and 1.0 mg/mL or less, 0.01 mg/mL or more and 1.0 mg/mL or less, 0.025 mg/mL or more and 1.0 mg/mL or less, and 0.025 mg/mL or more and 0.1 mg/mL or less. The extracellular matrix component may be dissolved in an appropriate solvent before use. Examples of such a solvent include, but are not limited to, water, buffer solutions, and aqueous acetic acid solutions. Among them, buffer solutions or aqueous acetic acid solutions are preferred. The pH of buffer solutions and aqueous acetic acid solutions is not particularly limited, as long as it does not adversely affect the cell growth and the formation of cell aggregates.

The term “polyelectrolyte” as used in the present specification refers to a polymer with a dissociable functional group in the polymer chain. As the polyelectrolyte used in the present embodiment, any polyelectrolyte can be used, as long as it does not adversely affect the cell growth and the formation of cell aggregates. Examples of the polyelectrolyte include, but are not limited to, glycosaminoglycans such as heparin, chondroitin sulfate (e.g., chondroitin 4-sulfate and chondroitin 6-sulfate), heparan sulfate, dermatan sulfate, keratan sulfate, and hyaluronic acid; dextran sulfate, rhamnan sulfate, fucoidan, carrageenan, polystyrene sulfonic acid, polyacrylamide-2-methylpropanesulfonic acid, polyacrylic acid, and derivatives thereof. These polyelectrolytes may be used singly or in combination of two or more.

The polyelectrolyte used in the present embodiment is preferably a glycosaminoglycan. In particular, heparin, chondroitin sulfate, or dermatan is preferable, and heparin is particularly preferable.

The concentration of the polyelectrolyte in the production method of the present embodiment is not particularly limited, as long as it does not adversely affect the cell growth and the formation of cell aggregates. The concentration of the polymer electrolyte may be more than 0 mg/mL and less than 1.0 mg/mL, 0.005 mg/mL or more and 1.0 mg/mL or less, 0.01 mg/mL or more and 1.0 mg/mL or less, 0.025 mg/mL or more and 1.0 mg/mL or less, and 0.025 mg/mL or more and 0.1 mg/mL or less.

The polyelectrolyte may be dissolved in an appropriate solvent before use. Examples of such a solvent include, but are not limited to, water and buffer solutions. When a cationic buffer solution is used as the cationic substance, the polyelectrolyte may be dissolved in the cationic buffer solution before use.

The mixing ratio (final concentration ratio) between the polyelectrolyte and the extracellular matrix component is preferably 1:2 to 2:1, and may be 1:1.5 to 1.5:1, or may be 1.1.

In step (A), a cell population comprising mouse-derived stromal cells (except for mouse-derived endothelial cells) and endothelial cells, a cationic substance, an extracellular matrix component, and a polyelectrolyte can be mixed in a suitable container, such as a dish, tube, flask, bottle, or plate. Mixture of these may be performed in a container used in step (B).

Subsequently, in step (B), a cell aggregate is obtained from the mixture obtained in step (A). The term “cell aggregate” as used in the present specification refers to a structure composed of cells combined into a single unit. The cell aggregate also includes cell precipitates obtained by centrifugation, filtration, or the like. In an embodiment, the cell aggregate is in the form of a slurry-like viscous body. The term “slurry-like viscous body” refers to a gel-like cell aggregate as described in Akihiro Nishiguchi et al., Cell-cell crosslinking by bio-molecular recognition of heparin-based layer-by-layer nanofilms, Macromol Biosci., 15 (3), 312-317, 2015.

A cell aggregate may be formed by placing the mixture obtained in step (A) in a suitable container and allowing it to stand. Alternatively, a cell aggregate may be formed by placing the mixture obtained in step (A) in a suitable container and collecting the cells by, for example, centrifugation, magnetic separation, or filtration. When the cells are collected by centrifugation, magnetic separation, filtration, or the like, the liquid part may or may not be removed.

The container used in step (B) may be a culture container used to culture cells. The culture container may be a container of material and shape which are commonly used for culturing cells and microorganisms. Examples of the material of the culture container include, but are not limited to, glass, stainless steel, plastic, and the like. Examples of the culture container include, but are not limited to, dishes, tubes, flasks, bottles, plates, and the like. At least part of the container is preferably formed of a material that allows liquid to pass through without allowing cells in the liquid to pass through. Examples of such containers include, but are not limited to, cell culture inserts, such as a Transwell (registered trademark) insert, Netwell (registered trademark) insert, Falcon (registered trademark) cell culture insert, and Millicell (registered trademark) cell culture insert.

The centrifugation conditions are not particularly limited, as long as they do not adversely affect the cell growth. For example, the cells may be collected by seeding the mixture in a cell culture insert and subjecting it to centrifugation at 10° C. and 400 ×g for 1 minute.

Subsequently, in step (C), the cell aggregate obtained in step (B) is cultured to obtain a three-dimensional cell structure. The cell culture in step (C) can be performed under culture conditions suitable for the cells to be cultured. A person skilled in the art can select a suitable medium depending on the cell type and desired function. Examples of the medium include, but are not limited to, DMEM, E-MEM, MEMα, RPMI-1640, McCoy's 5A, Ham's F-12, and the like, and media obtained by adding serum to these such that the serum constitutes about 1 to 20 volume % of the media. Examples of the serum include bovine serum (CS), fetal bovine serum (FBS), and fetal horse serum (HBS). Conditions such as the temperature and atmospheric composition of the culture environment may also be adjusted to the conditions suitable for the cells to be cultured.

Subsequently, in step (C), the cell aggregate obtained in step (B) is cultured to obtain a three-dimensional cell structure. The time required for culturing of a cell aggregate to obtain a three-dimensional cell structure may be 5 minutes to 168 hours, 12 hours to 144 hours, or 24 hours to 72 hours. Step (C) produces the effect of promoting adhesion between cells of the cell aggregate and thus providing a stable three-dimensional cell structure.

The cell aggregate may be suspended in a solution before culture. The solution is not particularly limited, as long as it does not adversely affect the cell growth and the formation of a three-dimensional cell structure. For example, media, buffer solutions, and the like suitable for cells that constitute the cell aggregate can be used. The cell aggregate can be suspended in a suitable container, such as a dish, tube, flask, bottle, or plate.

When the cell aggregate is suspended in a solution, the cells may be precipitated before culture to form cell precipitates. Precipitation of cells can be performed, for example, by centrifugation. The centrifugation conditions are not particularly limited, as long as they do not adversely affect the cell growth and the formation of a cell aggregate. For example, a suspension of a cell aggregate may be subjected to centrifugation at room temperature and 400 to 1,000 ×g for 1 minute to cause precipitation of cells. Alternatively, the cells may be precipitated by spontaneous sedimentation.

The container used in step (C) may be the same as the container used in step (B). In step (C), the container used in step (B) may be used as it is, or another container may be used.

During cell culture, substances for suppressing deformation of the constructed three-dimensional cell structure (e.g., contraction of tissue or detachment of tissue ends) may be added to the medium. Examples of such substances include, but are not limited to, Y-27632, which is a Rho-associated coiled-coil forming kinase/Rho binding kinase (ROCK) inhibitor.

Step (C) may be performed after steps (A) and (B) are performed in this order two or more times. By repeating steps (A) and (B), cell aggregates or cell precipitates can be laminated to produce a three-dimensional cell structure having a plurality of layers. That is, a three-dimensional cell structure having a large thickness can be produced.

In addition, when steps (A) and (B) are repeated to laminate cell aggregates or cell precipitates, a different cell population may be used for each repetition to laminate a three-dimensional cell structure composed of different types of cells. For example, after the first step (A) and step (B) are performed, the second step (A) is performed using a different cell population from the first step (A). Then, by performing the second step (B), a layer containing the cell population used in the second step (A) can be formed on the layer containing the cell population used in the first step (A). Multiple repetition of steps (A) and (B) leads to a lamination of a three-dimensional cell structure composed of a plurality of types of cell populations.

### <Three-Dimensional Cell Structure>

In an embodiment, the present invention provides a three-dimensional cell structure comprising a cell population comprising endothelial cells and mouse-derived stromal cells, the mouse-derived stromal cells excluding mouse-derived endothelial cells; a cationic substance; an extracellular matrix component; and a polyelectrolyte, wherein the ratio of the endothelial cells to the mouse-derived stromal cells (except for mouse-derived endothelial cells) in the cell population is 1.0% or more and 50% or less.

In the three-dimensional cell structure of the present embodiment, a slice obtained therefrom along a straight line passing through the center of gravity of the top surface thereof preferably has a maximum width of 40 μm or more. That is, a slice obtained from the above three-dimensional cell structure immediately after production along a straight line passing through the center of gravity of the top surface thereof preferably has a maximum width of 40 μm or more. The above maximum width immediately after production can be expressed in other words as described above.

Although the three-dimensional cell structure of the present embodiment is produced from mouse-derived cells, decrease in thickness thereof over time is suppressed. The extent of suppression of decrease in thickness of the three-dimensional cell structure of the present embodiment over time may be, for example, such that:

a first three-dimensional cell structure sample, which is selected from the one or more cell aggregates immediately after production as the one or more three-dimensional cell structures, has a first portion obtained therefrom as a first slice along a predetermined first straight line that passes through a first center of gravity of a top surface of the first three-dimensional cell structure sample;

a second three-dimensional cell structure sample, which is selected from the one or more cell aggregates on the fifth day since production as the one or more three-dimensional cell structures, has a second portion obtained therefrom as a second slice along a predetermined second straight line that passes through a second center of gravity of a top surface of the second three-dimensional cell structure sample;

and each of the first and second slices has a maximum width, the maximum width of the second slice being greater than or equal to 50% of that of the first slice. The above maximum width on the fifth day since production can be expressed in other words as described above.

In the three-dimensional cell structure of the present embodiment, the stromal cells, endothelial cells, cationic substance, extracellular matrix component, and polyelectrolyte are the same as those described above.

In the three-dimensional cell structure of the present embodiment, the ratio of endothelial cells to stromal cells (except for endothelial cells) in the cell population is preferably 1.0% or more and 50% or less, and may be 1.0% or more and 20% or less, or may be 1.0% or more and 10% or less.

### EXAMPLE

While the present invention will be described in greater detail referring to the examples, the present invention is not limited to these examples.

### Experimental Example 1

### <Production 1 of Three-Dimensional Cell Structure>

A three-dimensional cell structure was produced. As stromal cells, Mouse Embryonic Fibroblasts (MEF, available from ScienCell Research Laboratories, Inc.) were used. Further, as endothelial cells, mouse colon-derived vascular endothelial cells (available from Cell Biologics, Inc.) were used.

MEFs and the mouse colon-derived vascular endothelial cells were suspended at various ratios in different 50 mM tris-hydrochloric acid buffer solutions (pH 7.4) each containing 0.1 mg/mL heparin and 0.1 mg/mL collagen, to prepare cell suspensions where the ratio of endothelial cells to stromal cells was 0%, 1.5%, 3%, 4.5%, 10%, and 20% in respective suspensions. The collagen used was Collagen I.

Subsequently, the respective cell suspensions were centrifuged at 4° C. and 400 ×g for 3 minutes, the supernatant was removed, and were each resuspended in an appropriate amount of DMEM medium containing 10% fetal bovine serum (FBS). Each of the cell suspensions was then seeded in a 24-well cell culture insert such that each well contained 1.5×10⁶ MEFs. The bottom area per well of the cell culture insert was 33 mm².

Subsequently, the cell culture inserts were centrifuged at 4° C. and 400 ×g (gravitational acceleration) for 1 minute to obtain cell aggregates. A suitable amount of culture medium was then added to each cell culture insert, followed by culture in a CO₂ incubator (37° C., 5% CO₂) for one week. During this culture, medium replacement was appropriately performed.

After one day (when 24 hours has elapsed; that is, immediately after production) since the start of culture of the cell aggregates, and after four days (when 96 hours has elapsed; that is, on the fifth day since production) since the start of culture of the cell aggregates, each three-dimensional cell structure was fixed using 10% Mildform (FUJIFILM Wako Pure Chemical Corporation). Subsequently, each three-dimensional cell structure was embedded in paraffin, and slices of the respective three-dimensional cell structures were obtained along a straight line passing through the center of gravity as seen from the top surface thereof. Each of the slices was then subjected to hematoxylin and eosin (HE) staining and observed under a microscope to measure a maximum value of the thickness of each three-dimensional cell structure.

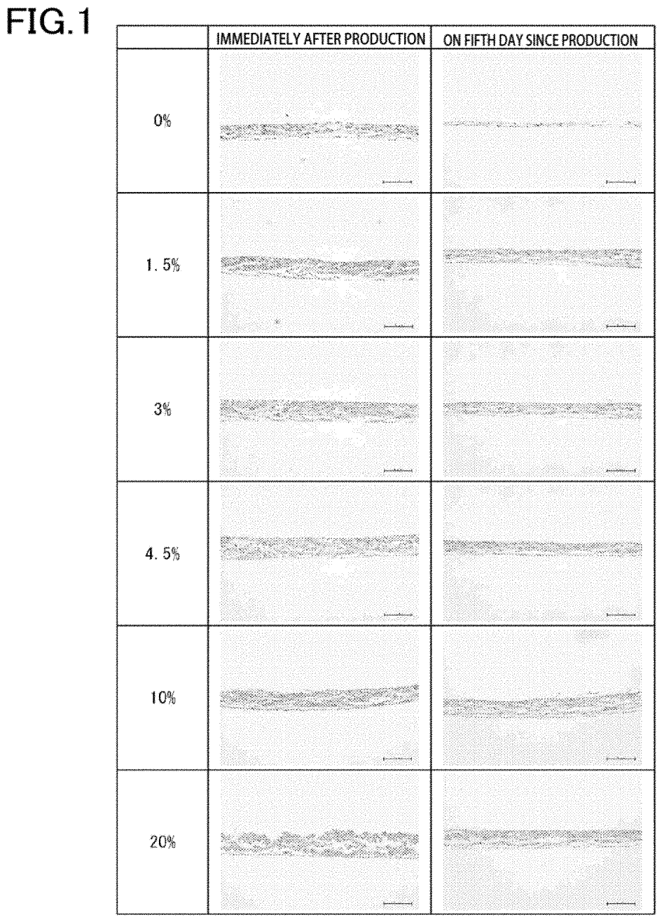

FIG. 1 shows fluorescence micrographs of slices of the respective three-dimensional cell structures. The scale bar represents 100 μm. In FIG. 1, the percentage (%) represents the ratio of endothelial cells to stromal cells used for production of the three-dimensional cell structure, and “IMMEDIATELY AFTER PRODUCTION” and “ON FIFTH DAY SINCE PRODUCTION” respectively indicate the three-dimensional cell structure fixed immediately after production of the cell aggregate and the three-dimensional cell structure fixed on the fifth day since production of the cell aggregate.

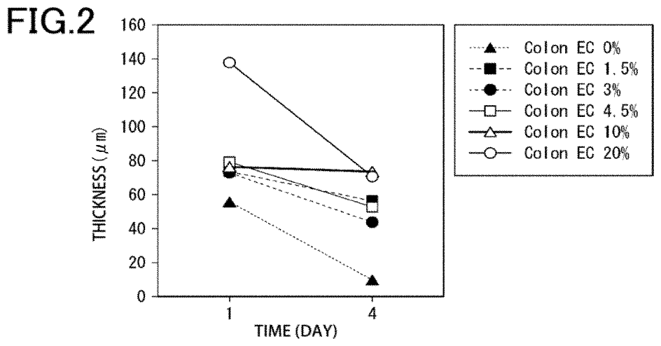

FIG. 2 is a graph showing the results of measuring maximum values of thicknesses of the respective three-dimensional cell structures based on FIG. 1. In FIG. 2, the vertical axis represents a maximum value of the thickness of the three-dimensional cell structure, whereas the horizontal axis represents the time (day) elapsed since culture of the cell aggregate started. Further, “colon EC” indicates mouse colon-derived vascular endothelial cells, and the percentage (%) represents the ratio of the number of endothelial cells to the number of stromal cells used for production of the three-dimensional cell structure. Table 1 shows the results of measuring maximum values of thicknesses of the respective three-dimensional cell structures immediately after production and on the fifth day since production.

TABLE 1

| | Thickness | | Ratio of thickness of |
|---|---|---|---|
| | of cell | Thickness of | cell structure on fifth |
| | structure | cell structure | day since production |
| Percentage of | immediately | on fifth day | to thickness of cell |
| endothelial | after produc- | since produc- | structure immediately |
| cells (%) | tion (μm) | tion (μm) | after production (%) |
| 0 | 55.7 | 9.6 | 17.2 |
| 1.5 | 73.5 | 56.2 | 76.5 |
| 3 | 72.8 | 43.6 | 59.9 |
| 4.5 | 79.3 | 52.6 | 66.4 |
| 10 | 76.3 | 73.5 | 96.3 |
| 20 | 137.8 | 70.6 | 51.2 |

It was revealed that the three-dimensional cell structure containing no endothelial cells markedly decreased in thickness on the fifth day since culture of the cell aggregate started. In contrast, it was revealed that, in each of the three-dimensional cell structures containing endothelial cells, decrease in thickness on the fifth day since culture of the cell aggregate started was markedly suppressed. Specifically, the three-dimensional cell structures, containing endothelial cells, immediately after production had a first portion obtained therefrom as a first slice along a predetermined first straight line that passes through a first center of gravity of a top surface thereof; the three-dimensional cell structures, containing endothelial cells, on the fifth day since production had a second portion obtained therefrom as a second slice along a predetermined second straight line that passes through a second center of gravity of a top surface thereof; and each of the first and second slices had a maximum width, the maximum width of the second slice being greater than or equal to 50% of that of the first slice.

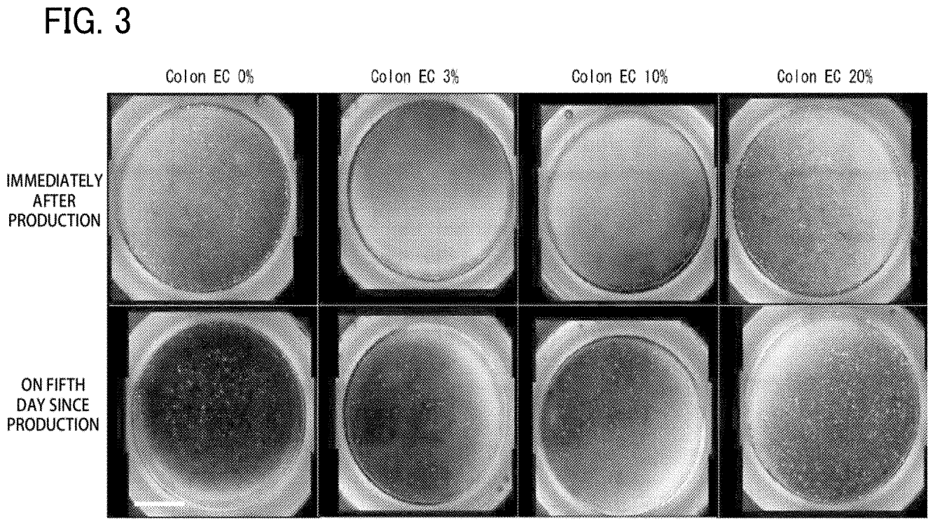

Further, vascular endothelial cells in the three-dimensional cell structures were stained with an anti-CD31 antibody and observed. FIG. 3 shows photographs showing the results of staining the respective three-dimensional cell structures with, as a first antibody, an anti-mouse CD31 rat antibody (Clone MEC 13.3, available from BD Biosciences), and then with, as a second antibody, an Alexa Fluor 488-labelled anti-rat IgG antibody (Thermo Fisher Scientific), and observing them under a fluorescence microscope. The scale bar represents 2 mm. As described above, the bottom area per well of the culture insert was 33 mm².

In FIG. 3, “colon EC” indicates mouse colon-derived vascular endothelial cells, and the percentage (%) represents the ratio of endothelial cells to stromal cells used for production of the three-dimensional cell structure. Further, “IMMEDIATELY AFTER PRODUCTION” and “ON FIFTH DAY SINCE PRODUCTION” respectively indicate results of the three-dimensional cell structures immediately after production of the cell aggregate and results thereof on the fifth day since production of the cell aggregate.

### Experimental Example 2

### <Production 2 of Three-Dimensional Cell Structure

Three-dimensional cell structures were produced as in Experimental Example 1 except that only stromal cells were used as cells. As stromal cells, normal human dermal fibroblasts (NHDF) were used. Specifically, NHDFs were suspended in 50 mM tris-hydrochloric acid buffer solutions (pH 7.4) containing 0.1 mg/mL heparin and 0.1 mg/mL collagen. The collagen used was Collagen I.

Subsequently, respective cell suspensions were centrifuged at 4° C. and 400 ×g for 3 minutes, the supernatant was removed, and they were each resuspended in an appropriate amount of DMEM medium containing 10% fetal bovine serum (FBS). Each of the cell suspensions was then seeded in a 24-well cell culture insert such that each well contained 2.0×10⁶ NHDFs. The bottom area per well of the cell culture insert was 33 mm².

Subsequently, the cell culture inserts were centrifuged at 4° C. and 400 ×g (gravitational acceleration) for 1 minute to obtain cell aggregates. A suitable amount of culture medium was then added to each cell culture insert, followed by culture in a CO₂ incubator (37° C., 5% CO₂) for one week. During this culture, medium replacement was appropriately performed.

After one day (when 24 hours has elapsed; that is, immediately after production) since the start of culture of the cell aggregates, and after five days (when 120 hours has elapsed; that is, on the sixth day since production) since the start of culture of the cell aggregates, each three-dimensional cell structure was fixed using 10% Mildform (FUJIFILM Wako Pure Chemical Corporation). Subsequently, each three-dimensional cell structure was embedded in paraffin, and slices of the respective three-dimensional cell structures were obtained along a straight line passing through the center of gravity as seen from the top surface thereof. Each of the slices was then subjected to hematoxylin and eosin (HE) staining and observed under a microscope to measure a maximum value of the thickness of each three-dimensional cell structure.

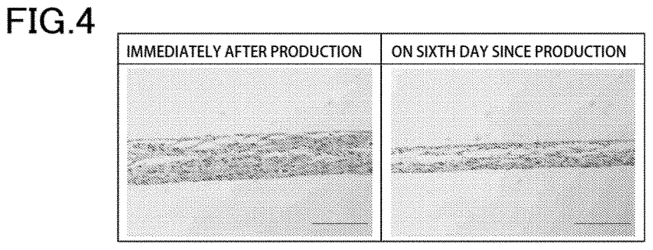

FIG. 4 shows fluorescence micrographs of slices of the three-dimensional cell structure. The scale bar represents 100 μm. In FIG. 4, “IMMEDIATELY AFTER PRODUCTION” and “ON FIFTH DAY SINCE PRODUCTION” respectively indicate the three-dimensional cell structure fixed immediately after production of the cell aggregate and the three-dimensional cell structure fixed on the fifth day since production of the cell aggregate.

As a result, the thickness of the three-dimensional cell structure immediately after production of the cell aggregate had a maximum value of 85.8 μm. Further, the thickness of the three-dimensional cell structure on the fifth day since production of the cell aggregate had a maximum value of 54.2 μm. This result revealed that, when a three-dimensional cell structure is produced using human-derived cells, the extent of decrease in thickness of the three-dimensional cell structure over time is markedly smaller than that when the three-dimensional cell structure is produced using mouse-derived cells.

The present application addresses the following. For example, cancer cells and immune cells may be cultured inside the three-dimensional cell structure or on the surface thereof, and the response of these immune cells to the cancer cells may be studied. In such a case, one way to eliminate the effects of rejection is to produce a three-dimensional cell structure using cells originating from an animal in the same strain, for example, cells originating from a mouse in the same strain, and culture mouse-derived cancer cells and immune cells inside the three-dimensional cell structure or on the surface thereof.

Unfortunately, the present inventors found that, when a three-dimensional cell structure is produced using mouse-derived cells, the thickness of the three-dimensional cell structure decreases over time. For example, in the case of a three-dimensional cell structure having a thickness of 50 μm immediately after production thereof, the three-dimensional cell structure may have a thickness of less than 10 μm on the fifth day since production thereof. Such a phenomenon is not observed when a three-dimensional cell structure is produced using cells of human origin.

Accordingly, an aspect of the present invention is to provide a technique for suppressing decrease in thickness of a three-dimensional cell structure over time when the three-dimensional cell structure is produced using mouse-derived cells.

The present invention includes the following aspects.

[1] A method for producing a three-dimensional cell structure, comprising:

a first step of performing a process of:
obtaining a mixture of a cell population, a cationic substance, an extracellular matrix component, and a polyelectrolyte, the cell population comprising mouse-derived stromal cells, the mouse-derived stromal cells excluding mouse-derived endothelial cells; and
collecting one or more cell aggregates from the obtained mixture; and

a second step of performing a culturing of the collected one or more cell aggregates to obtain one or more three-dimensional cell structures,

the cell population comprising endothelial cells in addition to the mouse-derived stromal cells.

[2] The production method according to [1], wherein:

a first three-dimensional cell structure sample, which is selected from the one or more cell aggregates immediately after production as the one or more three-dimensional cell structures, has a first portion cut away therefrom as a first slice along a predetermined first straight line that passes through a first center of gravity of a top surface of the first three-dimensional cell structure sample;

a second three-dimensional cell structure sample, which is selected from the one or more cell aggregates on the fifth day since production as the one or more three-dimensional cell structures, has a second portion cut away therefrom as a second slice along a predetermined second straight line that passes through a second center of gravity of a top surface of the second three-dimensional cell structure sample; and

each of the first and second slices has a maximum width, the maximum width of the second slice being greater than or equal to 50% of that of the first slice.

[3] The production method according to [1] or [2], wherein:

the first step repeatedly performs the process one or more times to collect cell aggregates as the one or more cell aggregates; and

the second step performs the culturing of each of the collected one or more cell aggregates.

[4] The production method according to any one of [1] to [3], wherein:

the extracellular matrix component is selected from collagen, laminin, fibronectin, vitronectin, elastin, tenascin, entactin, fibrillin, proteoglycan, and combinations thereof.

[5] The production method according to any one of [1] to [4], wherein:

a concentration of the extracellular matrix component in the mixture is 0.005 mg/mL or more and 1.0 mg/mL or less.

[6] The production method according to any one of [1] to [5], wherein:

the polyelectrolyte is selected from glycosaminoglycan, dextran sulfate, rhamnan sulfate, fucoidan, carrageenan, polystyrene sulfonic acid, polyacrylamide-2-methylpropanesulfonic acid, polyacrylic acid, and combinations thereof

[7] The production method according to any one of [1] to [6], wherein:

a concentration of the polyelectrolyte in the mixture is 0.005 mg/mL or more and 1.0 mg/mL or less.

[8] The production method according to any one of [1] to [7], wherein:

the mouse-derived stromal cells of the cell population are mouse-derived fibroblasts, and the endothelial cells of the cell population are vascular endothelial cells.

[9] The production method according to any one of [1] to [8], wherein:

a ratio of the endothelial cells to the stromal cells in the cell population is 1.0% or more and 50% or less.

[10] The production method according to any one of [1] to [9], wherein:

in the first step, the mixture is obtained in a liquid medium.

[11] A three-dimensional cell structure comprising:

a cell population comprising endothelial cells and mouse-derived stromal cells, the mouse-derived stromal cells excluding mouse-derived endothelial cells;

a cationic substance;

an extracellular matrix component; and

a polyelectrolyte,

wherein

a ratio of the endothelial cells to the mouse-derived stromal cells in the cell population is 1.0% or more and 50% or less.

[12] The three-dimensional cell structure according to [11],

the three-dimensional cell structure having a first portion cut away therefrom immediately after production as a first slice along a predetermined straight line that passes through a center of gravity of a top surface of the three-dimensional cell structure,

the first slice having a maximum width of 40 μm or more.

[13] The production method according to [11] or [12],

the three-dimensional cell structure having a second portion cut away therefrom on the fifth day since production as a second slice along a predetermined straight line that passes through the center of gravity of the top surface of the three-dimensional cell structure,

the second slice having a maximum width greater than or equal to 50% of the maximum width of the first slice.

### Industrial Applicability

The present application can provide a technique for suppressing decrease in thickness of a three-dimensional cell structure over time when the three-dimensional cell structure is produced using mouse-derived cells.

Obviously, numerous modifications and variations of the present invention are possible in light of the above teachings. It is therefore to be understood that within the scope of the appended claims, the invention may be practiced otherwise than as specifically described herein.

What is claimed is:

1. A method for producing a three-dimensional cell structure, comprising:
preparing a mixture of a cationic substance, an extracellular matrix component, a polyelectrolyte, and a cell population including a plurality of endothelial cells and a plurality of mouse-derived stromal cells, which exclude mouse-derived endothelial cells;
collecting a cell aggregate from the mixture; and
culturing a collected cell aggregate to obtain a three-dimensional cell structure.

2. The method according to claim 1, wherein a first three-dimensional cell structure sample, which is selected from the cell aggregate immediately after production as a three-dimensional cell structure, has a first portion cut away therefrom as a first slice along a predetermined first straight line that passes through a first center of gravity of a top surface of the first three-dimensional cell structure sample,
a second three-dimensional cell structure sample, which is selected from the cell aggregate on a fifth day since production as a three-dimensional cell structure, has a second portion cut away therefrom as a second slice along a predetermined second straight line that passes through a second center of gravity of a top surface of the second three-dimensional cell structure sample, and
the second slice has a maximum width being greater than or equal to 50% of a maximum width of the first slice.

3. The method according to claim 1, wherein the preparing and the collecting are conducted one or more times to collect a plurality of cell aggregates, and
the culturing is conducted on each of collected cell aggregates.

4. The method according to claim 1, wherein the extracellular matrix component comprises at least one selected from the group consisting of collagen, laminin, fibronectin, vitronectin, elastin, tenascin, entactin, fibrillin, and proteoglycan.

5. The method according to claim 1, wherein the mixture includes the extracellular matrix component at a concentration of 0.005 mg/mL-1.0 mg/mL.

6. The method according to claim 1, wherein the polyelectrolyte comprises at least one selected from the group consisting of glycosaminoglycan, dextran sulfate, rhamnan sulfate, fucoidan, carrageenan, polystyrene sulfonic acid, polyacrylamide-2-methylpropanesulfonic acid, and polyacrylic acid.

7. The method according to claim 1, wherein the mixture includes the polyelectrolyte at a concentration of 0.005 mg/mL-1.0 mg/mL.

8. The method according to claim 1, wherein the mouse-derived stromal cells of the cell population comprise mouse-derived fibroblasts, and the endothelial cells of the cell population comprise vascular endothelial cells.

9. The method according to claim 1, wherein a ratio of the endothelial cells to the stromal cells in the cell population is 1.0%-50%.

10. The method according to claim 1, wherein the preparing of the mixture produces the mixture in a liquid medium.

11. A three-dimensional cell structure, comprising:
a cell population comprising a plurality of endothelial cells and a plurality of mouse-derived stromal cells, the mouse-derived stromal cells excluding mouse-derived endothelial cells;
a cationic substance;
an extracellular matrix component; and
a polyelectrolyte,
wherein a ratio of the endothelial cells to the mouse-derived stromal cells in the cell population is 1.0%-50%.

12. The three-dimensional cell structure according to claim 11, wherein the three-dimensional cell structure includes a first portion cut away therefrom immediately after production as a first slice along a predetermined straight line that passes through a center of gravity of a top surface of the three-dimensional cell structure, and
the first slice has a maximum width of 40 μm or more.

13. The three-dimensional cell structure according to claim 11, wherein the three-dimensional cell structure includes a second portion cut away therefrom on a fifth day since production as a second slice along a predetermined straight line that passes through the center of gravity of a top surface of the three-dimensional cell structure, and
the second slice has a maximum width greater than or equal to 50% of the maximum width of the first slice.
